(19) United States
(12) Patent Application Publication
(10) Pub. No.: US 2010/0068341 A1
(43) Pub. Date: Mar. 18, 2010

(54) Granulates Containing Enzymes for Animal Food

(75) Inventor: Markus Lohscheidt, Heidelberg (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 11/991,834

(22) PCT Filed: Sep. 11, 2006
(86) PCT No.: PCT/EP2006/066217

§ 371 Date: Mar. 11, 2008

(30) Foreign Application Priority Data
Sep. 12, 2005 (DE) 10 2005 043 325.1

(51) Int. Cl. A23K 1/165; A23K 1/00; C12N 9/98; C12N 11/12; C12N 11/10

(52) U.S. Cl. 426/61

(57) ABSTRACT

The present invention relates to novel enzyme-comprising granules which are suitable as feed additives, and also to a method for production thereof. The invention also relates to the use of the enzyme-comprising granules in feed compositions and, in particular, pelleted feed compositions, which are obtainable using the enzyme-comprising granules.

The particles of the enzyme granules have

A. an enzyme-comprising core having a water content below 15% by weight, preferably in the range from 1 to 12% by weight, in particular in the range from 3 to 10% by weight, and especially in the range from 5 to 9% by weight, based on the weight of the enzyme-comprising core, which comprises
i) 50 to 96.9% by weight, preferably 55 to 94.85% by weight, and in particular 60 to 89.7% by weight, of at least one solid support material suitable for feeds,
ii) 0.1 to 10% by weight, preferably 0.15 to 5% by weight, in particular 0.2 to 2% by weight, and especially 0.3 to 1% by weight, of at least one water-soluble, neutral cellulose derivative,
iii) 3 to 49.9% by weight, frequently 5 to 49.85% by weight, in particular 10 to 44.8% by weight, and especially 10 to 39.7% by weight, of at least one enzyme, the weight fractions of i), ii) and iii) in each case being based on the nonaqueous components of the core; and

B) at least one hydrophobic coating arranged on the surface of the core, which coating comprises at least one hydrophobic material selected from waxes, saturated fatty acids, esters of saturated fatty acids, polyolefins and polyamides in an amount of at least 70% by weight, preferably at least 80% by weight, in particular at least 90% by weight, and especially at least 99% by weight, based on the total weight of the coating.

The present invention relates to novel enzyme-comprising granules which are suitable as feed additives, and also to a method for production thereof. The invention also relates to the use of the enzyme-comprising granules in feed compositions and, in particular, pelleted feed compositions, which are obtainable using the enzyme-comprising granules.

It is generally customary to add enzymes to animal feed in order to ensure better feed utilization, better product quality or lower pollution of the environment. In addition, it is current practice to supply animal feeds in pelleted form, since pelleting not only facilitates feed intake, but also improves handling of the feedstuff. In addition, it has been found that in the case of pelleted feedstuff, certain feed components are digested better, and ingredients added to the feedstuff such as, for example, vitamins, enzymes, trace elements, can be better incorporated in the feed mixture.

To reduce the microbial loading (sanitation) of such animal feeds, heat treating is frequently carried out. A heat treatment also proceeds in the context of the conditioning required for pelleting, in which the feedstuff is admixed with steam and thereby heated and moistened. In the actual pelleting step, the feedstuff is forced through a matrix. Other processes used in the feed industry are extrusion and expansion. The action of heat in all of these processes is in particular a problem, when enzymes, which are generally thermally unstable, are present in the feed mixture. Therefore, various efforts have been made to improve the thermal stability and, in particular, the pelleting stability of enzyme-comprising feed compositions.

There have been various proposals to improve the pelleting stability of enzyme granules by coating uncoated enzyme-comprising granules.

For instance, it is proposed in WO 92/12645 to provide enzyme-comprising granules which comprise 2 to 40% by weight cellulose fibers (what are termed T granules) with a coating which comprises a high fraction, preferably about 60 to 65% by weight, of an inorganic filler, such as, for example, kaolin, magnesium silicate or calcium carbonate. As follows from the examples of WO 92/12645, a single-stage application of the coating is not possible. Rather, it is necessary to apply in a plurality of steps alternately a high-melting fat or wax and the filler to the T granules. The disadvantages of the solution method proposed in this prior art for improving the pelleting stability are evident. Firstly, a very special support material is absolutely necessary, secondly complex multistage coating of the support material is necessary.

WO 2000/47060 describes enzyme-comprising granules which are suitable as feed additives and which have a polyethylene glycol coating.

WO 01/00042 teaches a method for coating enzyme granules with polymers. Coating agents which are proposed are aqueous solutions of polyalkylene oxide polymers, of homo- and copolymers of vinylpyrrolidone, of polyvinyl alcohols, and of hydroxypropylmethylcellulose, and also aqueous dispersions of alkyl (meth)acrylate polymers and polyvinyl acetate dispersions.

WO 03/059086 in turn teaches a method for producing enzyme granules having improved pelleting stability, in which enzyme-comprising raw granules are coated with an aqueous dispersion of a hydrophobic substance.

By means of the coating, the stability of the granules against decreasing enzyme activity can be basically improved, but the stabilities achieved are not completely satisfactory.

EP-A-0 257 996 proposes stabilizing enzymes for feed mixtures by pelleting them in a mixture with a support which has a main fraction of cereal flour.

WO 98/54980 in turn describes enzyme-comprising granules having improved pelleting stability which are produced by extruding an aqueous enzyme solution with a support based on an edible carbohydrate, and subsequent drying. Coating the granules is not described. The stability of these granules is not satisfactory.

PCT/EP 05/000826 in turn discloses improving the stability of the enzyme in liquid or solid enzyme formulations by adding gum Arabic or a plant protein to these.

It is therefore an object of the present invention to provide enzyme granules having improved enzyme stability, in particular pelleting stability. The enzyme granules should, in addition, be able to be produced in a simple manner and inexpensively. In addition, no losses in enzyme activity should occur even during the production.

It has surprisingly been found that enzyme granules having a hydrophobic coating exhibit particularly good pelleting stability particularly when the enzyme-comprising core which can still comprise up to 15% by weight of water, in addition to an enzyme, comprises at least one solid support material suitable for feeds in an amount of at least 50% by weight and 0.1 to 20% by weight of at least one water-soluble, neutral cellulose derivative, in each case based on the total amount of the nonaqueous components of the core.

The invention accordingly relates to enzyme granules for feeds, the particles of which
A. have an enzyme-comprising core having a water content below 15% by weight, preferably in the range from 1 to 12% by weight, in particular in the range from 3 to 10% by weight, and especially in the range from 5 to 9% by weight, based on the weight of the enzyme-comprising core which comprises
   i) 50 to 96.9% by weight, preferably 55 to 94.85% by weight, and in particular 60 to 89.7% by weight, of at least one solid support material suitable for feeds,
   ii) 0.1 to 10% by weight, preferably 0.15 to 5% by weight, in particular 0.2 to 2% by weight, and especially 0.3 to 1% by weight, of at least one water-soluble neutral cellulose derivative,
   iii) 3 to 49.9% by weight, frequently 5 to 49.85% by weight, in particular 10 to 44.8% by weight, and especially 10 to 39.7% by weight, of at least one enzyme,
   the percentages by weight of i), ii) and iii) in each case being based on the nonaqueous components of the core; and
B) have at least one hydrophobic coating arranged on the surface of the core, which coating comprises at least one hydrophobic material which is selected from waxes, saturated fatty acids, esters of saturated fatty acids, polyolefins and polyamides and is in an amount of at least 70% by weight, preferably at least 80% by weight, in particular at least 90% by weight, and especially at least 99% by weight, based on the total weight of the coating.

The inventive enzyme granules are distinguished by a particularly high stability, in particular a particularly high pelleting stability, and may be produced in a simple manner, the loss in enzyme activity during production being of the order of magnitude of comparable methods or below. Accordingly, the present invention also relates to the production method described here and to the use of the inventive enzyme granules in feed compositions, especially in pelleted feed compositions.

The granule particles of the inventive enzyme granules have an enzyme-comprising core and at least one hydrophobic coating arranged on the surface of the core. In addition, the granule particles can have one or more, for example 1, 2 or 3, further coatings of other materials, the coating of the inventive hydrophobic material preferably being arranged directly on the enzyme-comprising core.

Without wishing to be restricted to one theory, it may be assumed that the particular stability of the inventive enzyme granules is based on interaction of the inventive composition of the enzyme core with the inventive hydrophobic coating.

The weight ratio of core to hydrophobic coating is generally in the range from 70:30 to 99:1, preferably in the range from 75:25 to 98:2, in particular in the range from 80:20 to 96:4, and especially in the range from 85:15 to 95:5.

The inventive enzyme granules advantageously have a mean particle size (particle diameter) in the range from 100 to 2000 μm, in particular in the range from 200 to 1500 μm, and especially in the range from 300 to 1000 μm. The geometry of the granule particles is generally cylindrical having a ratio of diameter to length of about 1:1.3 to 1:3 and with if appropriate rounded ends. Typically, the particle sizes of the inventive coated enzyme granules correspond to those of the uncoated cores which hereinafter are also termed raw granules, that is the ratio of median particle diameter of the inventive granules to the median particle diameter of the raw granules generally not exceeding a value of 1.1:1, and in particular a value of 1.09:1.

As feed-compatible support materials, use can be made of customary inert inorganic or organic supports. An “inert” support must not exhibit any adverse interactions with the enzyme(s) of the inventive feed additive, such as, for example, cause irreversible inhibition of the enzyme activity, and must be harmless for use as an auxiliary in feed additives. Examples of suitable support materials which may be mentioned are: low-molecular-weight organic compounds, and also higher-molecular-weight organic compounds of natural or synthetic origin, and also inert inorganic salts. Preference is given to organic support materials. Among these, carbohydrates are particularly preferred.

Examples of suitable low-molecular-weight organic supports are, in particular, sugars such as, for example, glucose, fructose, sucrose. Examples of higher-molecular-weight organic supports which may be mentioned are carbohydrate polymers, in particular those which comprise α-D-glucopyranose, amylose or amylopectin units, in particular native and modified starches, microcrystalline cellulose, but also α-glucans and β-glucans, pectin (including protopectin) and glycogen. Preferably, the support material comprises at least one water-insoluble polymeric carbohydrate, in particular a native starch material such as, in particular, corn starch, rice starch, wheat starch, potato starch, starches of other plant sources such as starch from tapioca, cassava, sago, rye, oats, barley, sweet potatoes, arrowroot and the like, in addition cereal flours such as, for example, corn flour, wheat flour, rye flour, barley flour and oat flour, and also rice flour. Suitable materials are, in particular, also mixtures of the abovementioned support materials, in particular mixtures which predominantly, i.e. at least 50% by weight, based on the support material, comprise one or more starch materials. Preferably, the water-insoluble carbohydrate makes up at least 50% by weight, in particular at least 65% by weight, and especially at least 80% by weight, of the support material. Particularly preferred support materials are starches which comprise no more than 5% by weight, and in particular no more than 2% by weight, of protein or other components. A further preferred support material is microcrystalline cellulose. This can be used alone or in a mixture with the abovementioned support materials. If the microcrystalline cellulose is used in a mixture with other support materials, it preferably makes up no more than 50% by weight, in particular no more than 30% by weight, for example 1 to 50% by weight, in particular 1 to 30% by weight, and especially 1 to 10% by weight, of the support material.

Inorganic support materials which come into consideration are in principle all inorganic support materials known for feeds and feed additives, for example inert inorganic salts, for example sulfates or carbonates of alkali and alkaline earth metals such as sodium, magnesium, calcium and potassium sulfate or carbonate, in addition feed-compatible silicates such as talcum and silicic acids. The amount of inorganic support material, based on the total amount of support material, will generally not exceed 50% by weight, particularly 35% by weight, and very particularly 20% by weight. In a preferred embodiment, the organic support materials make up the total amount or virtually the total amount, that is at least 80% by weight, of the support material.

In addition to the feed-compatible support material, the enzyme-comprising core comprises according to the invention at least one water-soluble neutral cellulose derivative, in particular a cellulose ether. This cellulose derivative acts as binder and at the same time increases the pelleting stability compared with other known binders. Preferred cellulose derivatives have a number-average molecular weight in the range from 5×10³ to 5×10⁵ dalton, in particular in the range from 1×10⁴ to 2×10⁵ dalton. The cellulose derivatives are considered water-soluble when at least 3 g of cellulose derivative dissolves completely in 1 liter of water at 20° C.

Preferred cellulose derivatives are neutral cellulose ethers. Examples of inventively preferred water-soluble neutral cellulose ethers are methylcellulose, ethylcellulose and hydroxyalkylcelluloses, for example hydroxyethylcellulose (HEC), hydroxyethyl methylcellulose (HEMC), ethyl hydroxyethylcellulose (EHEC), hydroxypropylcellulose (HPC), hydroxypropyl methylcellulose (HPMC) and hydroxybutylcellulose. Among these, methylcellulose, ethylcellulose and mixed cellulose ethers having methyl groups or ethyl groups and hydroxyalkyl groups such as HEMC, EHEC and HPMC are particularly preferred. Preferred methyl- or ethyl-substituted cellulose ethers have a degree of substitution DS (with respect to the alkyl groups) in the range from 0.8 to 2.2 and in the case of mixed cellulose ethers, a degree of substitution DS with respect to alkyl groups in the range from 0.5 to 2.0, and a degree of substitution HS with respect to the hydroxyalkyl groups in the range from 0.02 to 1.0.

In a special embodiment of the invention, the water-soluble cellulose derivative comprises at least 50% by weight, in particular at least 80% by weight, in each case based on the total amount of cellulose derivatives, comprises one of the abovementioned neutral cellulose ethers, in particular methylcellulose or ethylcellulose, or mixed cellulose ethers having methyl groups or ethyl groups and hydroxyalkyl groups.

In addition, the enzyme-comprising core-forming material can also comprise water-soluble polymers different from cellulose derivatives, for example water-soluble proteins, for example proteins of animal origin such as gelatin, casein, in particular sodium caseinate, and plant proteins, such as soy protein, pea protein, bean protein, rapeseed protein, sunflower seed protein, cottonseed protein, potato protein, lupin, zein, wheat protein, corn protein and rice protein, synthetic polymers, for example polyethylene glycol, polyvinyl alcohol, and in particular the Kollidon brands from BASF, vinyl alcohol-vinyl ester copolymers, homo- and copolymers of vinylpyrrolidone with vinyl acetate and/or Cₗ-C₄-alkyl acrylates, if appropriate modified biopolymers, for example lignin, polylactide. The fraction of water-soluble polymers will preferably not exceed 50% by weight, in particular 20% by weight, in each case based on the total amount of cellulose derivative and water-soluble polymer, and is, where present, preferably in the range from 0.1 to 8% by weight, in particular 0.2 to 4% by weight, and especially 0.3 to 2% by weight, based on the total amount of the enzyme core-forming, nonaqueous components and is accordingly in these amounts also a component of the enzyme-comprising raw granules.

In addition, the enzyme core comprises at least one enzyme, mixtures of different enzymes also being able to be present. Typical enzymes for feeds are for example oxidoreductases, transferases, lyases, isomerases, ligases, lipases, and hydrolases. Examples of hydrolases, that is enzymes which cause a hydrolytic cleavage of chemical bonds, are esterases, glycosidases, keratinases, ether hydrolases, proteases, amidases, aminidases, nitrilases, and phosphatases. Glycosidases (EC 3.2.1, also termed carbohydrases) comprise not only endo- but also exoglycosidases, which cleave not only α- but also β-glycosidic bonds. Typical examples thereof are amylases, maltases, cellulases, endoxylanases, for example endo-1,4-β-xylanase or xylan endo-1,3-β-xylosidase, β-glucanases, in particular endo-1,4-β- and endo-1,3-β-glucanases, mannanases, lysozymes, galactosidases, pectinases, β-glucuronidases and the like. The inventive method is suitable in particular for producing pelleting-stable enzyme granules which are selected from non-starch polysaccharide-cleaving enzymes such as, for example, glucanases and xylanases, and also in particular from phosphatases (EC 3.1.3) and especially phytases (EC 3.1.3.8, 3.1.3.26 and 3.1.3.72).

The expression “phytase” comprises not only natural phytase enzymes, but also any other enzyme which exhibits phytase activity, for example is capable of catalyzing a reaction which liberates the phosphorus or phosphate from myoinositol phosphates. The phytase can be not only a 3-phytase (EC 3.1.3.8) but also a 4- or 6-phytase (EC 3.1.3.26) or a 5-phytase (EC 3.1.3.72) or a mixture thereof. Preferably, the phytase belongs to the enzyme class EC 3.1.3.8.

The enzyme used in the method according to the invention, in particular the phytase preferably used, is not subject to any restrictions and can be not only of microbiological origin, but also an enzyme obtained by genetic modification of a naturally occurring enzyme, or by de-novo construction. The phytase can be a phytase from plants, from fungi, from bacteria, or a phytase produced by yeasts. Preference is given to phytases from microbiological sources such as bacteria, yeasts or fungi. However, they can also be of plant origin. In a preferred embodiment, the phytase is a phytase from a fungal strain, in particular from an Aspergillus strain, for example Aspergillus niger, Aspergillus oryzae, Aspergillus ficuum, Aspergillus awamori, Aspergillus fumigatus, Aspergillus nidulans or Aspergillus terreus. Particular preference is given to phytases which are derived from a strain of Aspergillus niger or a strain of Aspergillus oryzae. In another preferred embodiment, the phytase is derived from a bacterial strain, in particular a Bacillus strain, an E. coli strain or a Pseudomonas strain, among these phytases being preferred which are derived from a Bacillus subtilis strain. In another preferred embodiment, the phytase is derived from a yeast, in particular a Kluveromyces strain or a Saccharomyces strain, among these phytases being preferred which are derived from a strain of Saccharomyces cerevisiae. In this invention, the expression “an enzyme derived from . . . ” comprises the enzyme naturally produced by the respective strain which is either obtained from the strain, or that is coded for by a DNA sequence isolated from the strain and is produced by a host organism which has been transformed using this DNA sequence. The phytase can be obtained from the respective microorganism by known techniques which typically comprise fermentation of the phytase-producing microorganism in a suitable nutrient medium (see, for example, ATCC catalog) and subsequently obtaining the phytase from the fermentation medium by standard techniques. Examples of phytases and of methods for preparing and isolating phytases may be found in EP-A 420358, EP-A 684313, EP-A 897010, EP-A 897985, EP-A 10420358, WO 94/03072, WO 98/54980, WO 98/55599, WO 99/49022, WO 00/43503, WO 03/102174, the contents of which are hereby explicitly incorporated by reference.

The amount of enzyme in the core obviously depends on the desired activity of the enzyme granules and the activity of the enzyme used and is typically in the range from 3 to 49.9% by weight, in particular in the range from 5 to 49.7% by weight, and especially in the range from 10 to 39% by weight, calculated as dry mass and based on the total weight of all nonaqueous components of the core material.

Phytase-comprising enzyme granules preferably have a phytase activity in the range from 1×10³ to 1×10⁵ FTU, in particular 5×10³ to 5×10⁴ FTU, and especially 1×10⁴ to 3×10⁴ FTU. 1 FTU of phytase activity is defined here as the amount of enzyme which releases 1 micromole of inorganic phosphate per minute from 0.0051 mol/l of aqueous sodium phytate at pH 5.5 and 37° C. The phytase activity can be determined, for example, according to “Determination of Phytase Activity in Feed by a Colorimetric Enzymatic Method”: Collaborative Interlaboratory Study Engelen et al.: Journal of AOAC International Vol. 84, No. 3, 2001, or else Simple and Rapid Determination of Phytase Activity, Engelen et al., Journal of AOAC International, Vol. 77, No. 3, 1994.

Enzyme granules which comprise an enzyme breaking down plant cell walls, for example a xylanase, typically have an enzyme activity in the range from 300 to 500 000, preferably 1000 to 250 000, in particular 1500 to 100 000, particularly preferably 2000 to 80 000, and especially 3000 to 70 000 EXU/g.

Enzyme granules which comprise a cellulase, for example a glucanase, such as a β-glucanase, typically have an enzyme activity in the range from 100 to 150 000, preferably 500 to 100 000, in particular 750 to 50 000, particularly preferably 1000 to 10 000, and especially 1500 to 8000 BGU/g.

Endo-xylanase activity (EXU) is defined as the amount of enzyme which releases 1.00 micromoles of reducing sugar, measured as xylose equivalents per minute at pH 3.5 and 40° C. One beta-glucanase unit (BGU) is defined as the amount of enzyme which releases 0.258 micromole of reducing sugar, measured as glucose equivalents per minute, at pH 3.5 and 40° C. Endo-xylanase activity (EXU) and p-glucanase activity

(BGU) can be determined according to Engelen et al.: Journal of AOAC International Vol. 79, No. 5, 1019 (1996).

In addition, the core-forming material can additionally comprise a salt stabilizing the enzyme. Stabilizing salts are typically salts of divalent cations, in particular salts of calcium, magnesium or zinc, and also salts of monovalent cations, in particular sodium or potassium, for example the sulfates, carbonates, hydrogencarbonates and phosphates including hydrogenphosphates and ammonium hydrogenphosphates of these metals. Preferred salts are sulfates. Particular preference is given to magnesium sulfate and zinc sulfate, including their hydrates. The amount of salt is preferably in the range from 0.1 to 10% by weight, in particular in the range from 0.2 to 5% by weight, and especially in the range from 0.3 to 3% by weight, based on the total weight of all nonaqueous components of the core material.

In addition, the core can comprise further components in subsidiary amounts which generally make up no more than 10% by weight, in particular no more than 5% by weight, of the core, based on its dry components (that is nonaqueous components), for example means for setting the pH such as buffers (phosphate buffer, potassium phosphate or sodium phosphate, hydrates or dihydrates thereof, sodium carbonate or potassium carbonate, acetate, propionate, tartrate, hydrogencarbonate, phthalate, hydrogen phthalate, in particular the sodium, potassium or calcium salts of the abovementioned substances including their hydrates or dihydrates), bases (sodium, potassium, calcium, magnesium, ammonium hydroxide, ammonia water) or acids (inorganic or organic acids, hydrochloric acid, sulfuric acid, phosphoric acid, citric acid, acetic acid, formic acid, propionic acid).

The particles of the inventive enzyme granules in addition have at least one hydrophobic coating arranged on the core of the particles. The hydrophobic coating will preferably at least 80% (mean value) and particularly completely cover the surface of the cores.

Hydrophobic materials which come into consideration for the hydrophobic coating are not only polymeric substances but also oligomeric or low-molecular-weight substances. According to the invention, the hydrophobic materials have a high hydrocarbon fraction, the fraction of carbon and hydrogen generally making up at least 80% by weight, in particular at least 85% by weight, of the hydrophobic material. Belonging to those substances are substances which have a melting point above 30° C., more preferably above 40° C., in particular above 45° C., and especially above 50° C., or in the case of non-melting substances are solid at these temperatures or have a glass transition temperature above these temperatures. Preference is given to hydrophobic materials having melting points in the range from 40 to 95° C., in particular in the range from 45 to 80° C., and particularly preferably in the range from 50 to 70° C.

Preferably, the hydrophobic material is low-acid, and has an acid value less than 80, in particular less than 30, and especially less than 10 (determined as specified in ISO 660).

Examples of hydrophobic materials suitable according to the invention are
polyolefins such as polyethylene, polypropylene and polybutenes;
saturated fatty acids preferably having 10 to 32 carbon atoms, frequently 12 to 24 carbon atoms, and in particular 16 to 22 carbon atoms;
esters of saturated fatty acids, preferably mono-, di- and triglycerides and also esters of saturated fatty acids with fatty alcohols. The fatty alcohols for example have 10 to 32 carbon atoms, in particular 16 to 24 carbon atoms, such as cetyl alcohol or stearyl alcohol. The fatty acids and the fatty acid radicals in the esters of fatty acids preferably have 10 to 32 carbon atoms, frequently 12 to 24 carbon atoms and, in particular, 16 to 22 carbon atoms;
polyamides
waxes, in particular plant waxes and waxes of animal origin, but also montan waxes and montan ester waxes and also microcrystalline waxes, for example waxes based on saturated hydrocarbons (isoalkanes), alkyl-substituted cycloparaffins and alkyl-substituted and naphthene-substituted aromatics.

In a preferred embodiment, the coating-forming material comprises up to at least 70% by weight, particularly up to at least 80% by weight, in particular up to at least 90% by weight, of at least one substance selected from saturated fatty acids, esters of fatty acids and mixtures thereof, esters of fatty acids and, in particular, triglycerides being preferred. Saturated means that the hydrophobic material is essentially free from unsaturated components and correspondingly has an iodine value less than 5 and, in particular, less than 2 (method according to Wijs, DIN 53 241).

Particularly preferably, the coating comprises up to at least 70% by weight, in particular at least 80% by weight, and especially at least 90% by weight, of the abovementioned triglycerides.

In a preferred embodiment of the invention, the coating agent predominantly, that is up to at least 70% by weight, in particular at least 80% by weight, and especially greater than 90% by weight, comprises hydrogenated vegetable oils, in particular triglycerides of plant origin, for example hydrogenated cottonseed, corn, peanut, soybean, palm, palm kernel, babassu, rapeseed, sunflower and safflower oils. Hydrogenated vegetable oils which are particularly preferred among these are hydrogenated palm oil, cottonseed oil and soybean oil. The most preferred hydrogenated vegetable oil is hydrogenated soybean oil. Similarly, other fats and waxes originating from plants and animals are also suitable, for example beef tallow. Suitable materials are also nature-identical fats and waxes, that is synthetic waxes and fats having a composition which predominantly corresponds to that of the natural products.

The table below mentions some examples of coating materials which are suitable according to the invention:

| | | Melting | |
|---|---|---|---|
| Name | Composition | range | CAS No./INCI |
| Cutina CP from | synthetic cetyl palmitate | 46-51° C. | 95912-87-1 |
| Cognis | | | cetyl palmitate |
| Edenor NHTI-G | Triglyceride | 56-60° C. | 67701-27-3* |
| from | | | |
| Cognis | | | |
| Edenor NHTI-V from | Triglyceride | 57-60° C. | 67701-27-3* |
| Cognis | | | EINECS 266-945-8 |
| Edenor C1892 from | Stearic acid, C16-18 | 66-99° C. | |
| Cognis | | | |
| Edenor HPA from | Fatty acids, palm oil, | 55-57° C. | |
| Cognis | hydrogenated, | | |
| | C16-18 | | |
| Edenor HRAGW | Fatty acids, C16-22 | 64-66° C. | |
| from Cognis | | | |
| Edenor C2285R | Fatty acids, C18-22 | 75-78° C. | 68002-88-0* |
| from Cognis | | | |
| Rilanit from Cognis | Triglyceride | 83-90° C. | |
| Japan wax | principally glycerol palmitate | 49-55° C. | rhus succedanea |
| substitute Kahl - | | | |
| Wachsraffinerie | | | |
| Tefacid from | palm kernel oil Tefacid Palmic 90 | 65° C. | 57-10-3 |
| Karlshamns | | | |
| Soybean fat powder | | 65-70° C. | |
| from Sankyu/Japan | | | |

Suitable products are also those of the company Aarhus Olie, Denmark, marketed under the trademark Vegeol PR, for example Vegeol® PR 267, PR 272, PR 273, PR 274, PR 275, PR 276, PR 277, PR 278 and PR 279.

Waxes suitable as coating materials are, in particular, waxes of animal origin such as beeswax and lanolin, waxes of plant origin such as candelilla wax, carnauba wax, cane sugar wax, caranday wax, raffia wax, Columbia wax, esparto wax, alfalfa wax, bamboo wax, hemp wax, Douglas fir wax, cork wax, sisal wax, flax wax, cotton wax, dammar wax, cereal wax, rice wax, ocatilla wax, oleander wax, montan waxes, montan ester waxes, polyethylene waxes, in addition the products of Süddeutsche Emulsions-Chemie marketed under the trademarks Wükonil, Südranol, Lubranil or Mikronil, or the BASF products having the trademarks Poligen WE1, WE3, WE4, WE6, WE7, WE8 BW, WE9.

Of course, the inventive enzyme granules, in addition to the hydrophobic coating, can also have one or more, for example, 1, 2 or 3, further coatings which comprise other materials, for example the coatings taught in the prior art. It is essential to the invention that at least one coating consists of the hydrophobic materials, this layer being able to be arranged as desired and, in particular, arranged directly on the enzyme-comprising core.

The inventive enzyme granules can be produced by analogy with known production methods for coated enzyme granules, for example analogously to the procedures described in WO 01/00042, WO 03/059086 or PCT/EP 2005/000826.

According to a preferred embodiment, the method comprises the following steps:
a) providing uncoated, enzyme-comprising raw granules, preferably having a water content below 15% by weight, frequently in the range from 1 to 12% by weight, in particular in the range from 3 to 10% by weight, and especially in the range from 5 to 9% by weight, based on the weight of the enzyme-comprising raw granules,
b) applying the hydrophobic coating to the particles of the raw granules.

The raw granules can be produced in principle in any desired manner. For example, a mixture comprising the feed-compatible support, at least one water-soluble, neutral cellulose derivative, and at least one enzyme and if appropriate further components such as water, buffer, stabilizing metal salts, can be processed to form raw granules in a manner known per se by extrusion, mixer-granulation, fluidized-bed granulation, disk agglomeration or compacting.

In a preferred embodiment, production of the raw granules comprises in a first step the extrusion of a water-comprising dough which comprises at least one water-soluble, neutral cellulose derivative and at least one enzyme and if appropriate further components such as water, buffer, stabilizing metal salts in the amounts stated above.

The support material generally accordingly makes up 50 to 96.9% by weight, preferably 55 to 94.8% by weight, and in particular 60 to 89.7% by weight of the nonaqueous components of the dough. The at least one, water-soluble, neutral cellulose derivative generally makes up 0.1 to 10% by weight, preferably 0.15 to 5% by weight, in particular 0.2 to 2% by weight, and especially 0.3 to 1% by weight, of the nonaqueous components of the dough. The at least one enzyme generally makes up 3 to 49.9% by weight, in particular 5 to 49.85% by weight, in particular 10 to 44.8, and especially 10 to 39.7% by weight, of the nonaqueous components of the dough. The fraction of other components corresponds to the weight fractions given above for the composition of the core.

In addition to aforesaid components, the dough comprises water in an amount which ensures sufficient homogenization for the dough-forming components and adequate consistency (plasticization) of the dough for extrusion. The amount of water required for this can be determined in a manner known per se by those skilled in the art in the field of enzyme formulation. The water fraction in the dough is typically in the range from >15 to 50% by weight, in particular in the range from 20 to 45% by weight, and especially in the range from 25 to 40% by weight, based on the total weight of the dough.

The dough is produced in a manner known per se by mixing the dough-forming components in a suitable mixing apparatus, for example in a conventional mixer or kneader. For this, the solid or solids, for example the support material, are intensively mixed with the liquid phase, for example water, an aqueous binder solution, or an aqueous enzyme concentrate. Generally, the support will be introduced as solid into the mixer and mixed with an aqueous enzyme concentrate and also with the water-soluble polymer, preferably in the form of a separate aqueous solution or dissolved in the aqueous enzyme concentrate, and also if appropriate with the stabilizing salt, preferably in the form of a separate aqueous solution or suspension, in particular dissolved or suspended in the aqueous enzyme concentrate. If appropriate, further water will be added to set the desired consistency of the dough. Preferably, during mixing, a temperature of 60° C., in particular 40° C., will not be exceeded. Particularly preferably, the temperature of the dough during mixing is 10 to 30° C. If appropriate, therefore, the mixing apparatus will be cooled during dough production.

In addition, it has proved useful to control the pH of the aqueous phase before or during the dough production. According to a preferred embodiment of the invention, therefore a pH will be established in the range from 3.5 to 7, in particular in the range from 4 to 6, and especially in the range from 4.5 to 5.5. Setting the pH leads surprisingly likewise to an improved stability of the enzyme granules, in particular when the enzyme is a hydrolase, and especially a phosphatase. For setting the pH, use can be made of, for example, an acid or base, or a buffer. Preferably, those agents for setting the pH will be selected which are permitted in feeds. The agent for setting the pH can be added either to the dough as such, or together with one of the abovementioned components of the dough, preferably in the form of an aqueous solution. In particular, the agent for setting the pH is added in a form dissolved in the enzyme concentrate. Accordingly, the pH of the enzyme concentrate is set in accordance with the abovementioned ranges preferably before mixing. The agent for setting the pH naturally depends on the pH which is set during mixing of the components. Since the enzyme concentrate frequently has a weakly acidic pH less than 4, preferably a buffer or a base will be added. Suitable bases are, in addition to ammonia, ammonia water and ammonium hydroxide, alkali metal and alkaline earth metal hydroxides, citrates, acetates, formates, hydrogenformates, carbonates and hydrogencarbonates and also amines and alkaline earth metal oxides such as CaO and MgO. Examples of inorganic buffering agents are alkali metal hydrogenphosphates, in particular sodium and potassium hydrogenphosphates, for example K₂HPO₄, KH₂PO₄ and Na₂HPO₄.

A preferred agent for setting the pH is ammonia or ammonia water, or sulfuric acid. Suitable buffers are, for example, mixtures of the abovementioned bases with organic acids such as acetic acid, formic acid, citric acid.

The resultant dough is subsequently subjected to an extrusion, preferably an extrusion at low pressure. The extrusion, in particular extrusion at low pressure, generally proceeds in an apparatus in which the mix (dough) to be extruded is forced through a matrix. The hole diameter of the matrix determines the particle diameter and is generally in the range from 0.3 to 2 mm, and in particular in the range from 0.4 to 1.0 mm. Suitable extruders are, for example, dome extruders or basket extruders which, inter alia, are marketed by companies such as Fitzpatrick or Bepex. For correct consistency of the mix to be granulated, in this case only a low temperature increase results on passing through the matrix (up to approximately 20° C.). Preferably, the extrusion proceeds under temperature control, that is the temperature of the dough should not exceed a temperature of 70° C., in particular 60° C., during extrusion. In particular, the temperature of the dough during extrusion is in the range from 20 to 50° C.

The extruded dough strands leaving the extruder break up into short granule-like particles or can be broken if appropriate using suitable cutting apparatuses. The resultant granule particles typically have a homogeneous particle size, that is a narrow particle size distribution.

In this manner raw granules are obtained having a comparatively high water content which is generally greater than 15% by weight, for example in the range from 15 to 50% by weight, in particular in the range from 20 to 45% by weight, based on the total weight of the moist raw granules. According to the invention, therefore, before coating, processing is carried out in such a manner that the water content of the raw granules is no greater than 15% by weight and preferably is in the range from 1 to 12% by weight, in particular in the range from 3 to 10% by weight, and especially in the range from 5 to 9% by weight.

The final processing therefore generally comprises a drying step. This preferably proceeds in a fluidized-bed dryer. In this case, a heated gas, generally air or a nitrogen gas stream, is passed from below through the product layer. The gas rate is customarily set so that the particles are fluidized and swirl. As a result of the gas/particles heat transfer, the water evaporates. Since enzyme-comprising raw granules are generally heat-labile, it is necessary to ensure that the temperature of the raw granules does not rise too high, that is generally not above 80° C., and preferably not above 70° C. In particular, the temperature of the granules during drying is in the range from 30 to 70° C. The drying temperature can be controlled in a simple manner via the temperature of the gas stream. The temperature of the gas stream is typically in the range from 140 to 40° C., and in particular in the range from 120 to 60° C. Drying can proceed continuously or batchwise.

After drying, the granules can be further fractionated by means of a sieve (optional). Coarse material and fines can be ground and returned to the mixer for preparing the granulation mix.

In addition, it has proved to be advantageous to round, that is to say spheronize, the still-moist raw granules before carrying out drying. In this case, in particular, the formation of unwanted dust fractions in the end product is decreased.

Apparatuses suitable for rounding the moist raw granules are what are termed spheronizers which essentially have a horizontally rotating disk on which the small extruded rods are forced to the wall by the centrifugal force. The small extruded rods break up on the micronotches prefixed by the extrusion process, so that cylindrical particles are formed having a ratio of diameter to length of about 1:1.3 to 1:3. As a result of the mechanical load in the spheronizer, the initially cylindrical particles are somewhat rounded.

The raw granules obtained after final processing advantageously have a median particle size in the range from 100 to 2000 μm, in particular in the range from 200 to 1500 μm, and especially in the range from 300 to 1000 μm. The median particle size distribution can be determined in a manner known per se by light scattering, for example using a Mastersizer S from Malvern Instruments GmbH or by sieve analysis, for example using a Vibro VS 10000 sieving machine from Retsch. The median particle size is taken by those skilled in the art to mean the D₅₀ value of the particle size distribution curve, that is to say the value which 50% by weight of all particles fall above or below. Preference is given to raw granules having a narrow particle size distribution.

The amount of hydrophobic material is generally 1 to 35% by weight, preferably 4 to 30% by weight, in particular 5 to 25% by weight, and especially 7 to 21% by weight, based on the raw granules used and dried.

The hydrophobic material can be applied in a manner known per se by application of a solution, dispersion or suspension of the hydrophobic material in a suitable solvent, for example water, or by application of a melt of the hydrophobic material. The application of a melt is preferred according to the invention, because the subsequent removal of solvent or dispersion medium can thereby be avoided. This means that for application of a melt, the use of an expensive dryer/coater (for example a fluidized-bed dryer) is not required, but the use of a mixer is possible. Coating with a melt of the hydrophobic material is also termed hereinafter melt coating.

Suitable methods for applying the coating comprise coating in a fluidized bed, and also coating in a mixer (continuously or batchwise), for example a granulation drum, a ploughshare mixer, for example from Lodige, a paddle mixer, for example from Forberg, a Nauta mixer, a granulating mixer, a granulating dryer, a vacuum coater, for example from Forberg, or a high-shear granulator.

In particular, the raw granules are coated
i) in a fluidized bed, for example by spraying the raw granules with a melt, a solution or dispersion of the hydrophobic material; and also
ii) in one of the abovementioned mixing apparatuses by introduction of the raw granules into a melt of the hydrophobic material or by spraying or dousing the raw granules with a melt, a solution or dispersion of the hydrophobic material.

Coating the raw granules by spraying with a melt, a solution or dispersion in a fluidized bed is particularly preferred according to the invention. Spraying the raw granules with a melt, a solution or dispersion of the hydrophobic material can be carried out in the fluidized-bed apparatus in principle in the bottom-spray method (nozzle is seated in the gas-distribution plate and sprays upwards) or in the top-spray method (coating is sprayed into the fluidized bed from the top).

The raw granules can be coated in the context of the inventive method continuously or batchwise.

According to a first preferred embodiment of the inventive method, the raw granules are charged into a fluidized bed, swirled and, by spraying on an aqueous or nonaqueous, preferably aqueous, dispersion of the hydrophobic material, are coated with this material. For this use is made of preferably a liquid which is as highly concentrated as possible and still sprayable, such as, for example, a 10 to 50% strength by weight aqueous dispersion or nonaqueous solution or dispersion of the hydrophobic material.

The solution or dispersion of the hydrophobic material is preferably sprayed on in such a manner that the raw granules are charged into a fluidized-bed apparatus or a mixer and sprayed onto the spray material with simultaneous heating of the charge. The energy is supplied in the fluidized-bed apparatus by contact with heated drying gas, frequently air. Preheating the solution or dispersion can be expedient when as a result spray material having a higher dry substance fraction can be sprayed. When use is made of organic liquid phases, solvent recovery is expedient and the use of nitrogen as drying gas to avoid explosive gas mixtures is preferred. The product temperature during coating should be in the range from about 30 to 80° C., and in particular in the range from 35 to 70° C., and especially in the range from 40 to 60° C. Coating can be carried out in the fluidized-bed apparatus in principle in the bottom-spray method (nozzle is seated in the gas-distribution plate and sprays upwards) or in the top-spray method (coating is sprayed into the fluidized bed from the top). When a mixer is used for coating, after the solution or dispersion is sprayed on, the solvent or the liquid of the dispersion must be removed. This can be carried out in a dryer.

According to a second, particularly preferred embodiment of the inventive method, the raw granules charged into a fluidized bed or mixer are coated with a melt of the hydrophobic material. Melt coating in a fluidized bed is preferably carried out in such a manner that the raw granules to be coated are charged into the fluidized-bed apparatus. The hydrophobic material is melted in an external reservoir and pumped, for example via a heatable line to the spraying nozzle. Heating the nozzle gas is expedient. Spraying rate and inlet temperature of the melt are preferably set in such a manner that the hydrophobic material still runs readily on the surface of the granules and evenly coats them. Preheating the granules before spraying the melts is possible. In the case of hydrophobic materials having a high melting point, generally the temperature will be selected in such a manner that a loss of enzyme activity is substantially avoided. The product temperature should therefore preferably be in the range from about 30 to 80° C., and in particular in the range from 35 to 70° C., and especially in the range from 40 to 60° C. Melt coating can also be carried out in principle by the bottom-spray method or by the top-spray method.

Melt coating can be carried out in a mixer in two different ways. Either the granules to be coated are charged into a suitable mixer and a melt of the hydrophobic material is sprayed or poured into the mixer. Another possibility is to mix the hydrophobic material present in solid form with the product. By supplying energy via the vessel wall or via the mixing tools, the hydrophobic material is melted and thus coats the raw granules. According to requirement, from time to time a little release agent can be added. Suitable release agents are, for example, silicic acid, talcum, stearates and tricalcium phosphate, or salts such as magnesium sulfate, sodium sulfate or calcium carbonate.

The solutions, dispersions or melts used for coating can, if appropriate, be admixed with other additives, such as, for example, microcrystalline cellulose, talcum and kaolin, or salts.

In a particular inventive embodiment of the method, the addition of release agents during application of the hydrophobic material or the addition of release agents to the solution, dispersion or melt to be applied can be omitted. This is possible, in particular, when the enzyme cores used have median particle sizes of at least 300 μm, preferably at least 350 μm, in particular at least 400 μm, for example in the range from 250 to 1600 μm, preferably in the range from 300 μm to 1500 μm, and in particular in the range from 400 μm to 1400 μm, and simultaneously the amount of hydrophobic coating material used based on the total particle is no greater than 30% by weight, preferably no greater than 25% by weight, in particular no greater than 20% by weight, and especially no greater than 17% by weight. In these cases, enzyme cores may be coated particularly readily without agglomeration of the particles.

The addition of a flow aid after the coating step can enhance the flow properties of the product. Typical flow aids are silicic acids, for example the Sipernat products from Degussa or the Tixosil products from Rhodia, talcum, stearates and tricalcium phosphate, or salts such as magnesium sulfate, sodium sulfate or calcium carbonate. The flow aids are added to the coated product in an amount of from 0.005% by weight to 5% by weight based on the total weight of the product. Preferred contents are 0.1% by weight to 3% by weight, and particularly preferred 0.2% by weight to 1.5% by weight.

The invention further relates to feed compositions, and in particular pelleted feed compositions which, in addition to customary components, comprise at least one feed additive in accordance with the above definition as admixture.

Finally, the invention also relates to the use of a feed additive according to the above definition for producing feed compositions, in particular hydrothermally treated, and especially pelleted, feed compositions.

For production of the feed compositions, the coated enzyme granules produced according to the invention are mixed with conventional animal feed (such as, for example, pig-fattening feed, piglet feed, sow feed, broiler feed and turkey feed). The enzyme granule fraction is selected in such a way that the enzyme content is, for example, in the range from 10 to 1000 ppm. Subsequently, the feed is pelleted using a suitable pellet press. For this the feed mixture is customarily conditioned by steam introduction and subsequently pressed through a matrix. Depending on the matrix, pellets of about 2 to 8 mm in diameter can be produced in this way. The highest process temperature occurs in this case during conditioning or during pressing of the mixture through the matrix. Here, temperatures in the range from about 60 to 100° C. can be reached.

### COMPARATIVE EXAMPLE C1

a) In an aqueous phytase concentrate having a dry mass content of about 25 to 35% by weight, a pH in the range of 3.7-3.9 and an activity of 26 000 to 36 000 FTU/g, 1% by weight of zinc sulfate hexahydrate, based on the concentrate, was dissolved at 4-10° C.
b) In a mixer having a chopper blade, 900 g of corn starch were charged, homogenized and to this were added slowly at temperatures of 10 to 30° C. with homogenization simultaneously 380 g of the zinc sulfate-comprising phytase concentrate and 140 g of a 10% strength by weight solution of polyvinyl alcohol (degree of hydrolysis: 87-89%). The mixture was homogenized with cooling of the mixer for a further 5 min at temperatures in the range from 10 to 50° C., then the resultant dough was transferred to a dome extruder and the dough was extruded at temperatures in the range from 30 to 50° C. through a matrix having an orifice diameter of 0.7 mm to give 5 cm long strands.
c) The resultant extrudate was rounded in a rounding machine (type P50, from

Glatt) for 5 min. at 350 min⁻¹ (speed of rotation of the rotating disk) and then dried to a residual moisture of about 6% by weight in a fluidized-bed dryer at a temperature of up to 40° C. (product temperature).

The resultant raw granules had an activity of approximately 14 200 FTU/g. The granules had a particle size of a maximum of 1300 μm and median particle size of 650 μm (sieve analysis).
d) For the subsequent coating, the raw granules were charged into a laboratory fluidized bed Aeromat type MP-1 from Niro-Aeromatic. As receiving vessel, use was made of a plastic cone having a gas-distribution plate diameter of 110 mm and a perforated plate having 12% open area. The coating agent was a commercially available triglyceride based on saturated C₁₆/C₁₈ sfatty acids (melting point 57-61° C., iodine value 0.35, saponificaton value 192).

The raw granules (700 g) charged into the fluidized bed were heated to a product temperature of 45° C. with swirling using an air rate of 50 m³/h. 124 g of the triglyceride were melted in a glass beaker at 85° C. and sprayed onto the raw granules by means of a two-fluid nozzle (1 mm) in the bottom-spray method by reduced-pressure suction at 1 bar spraying pressure using heated spraying gas from 80 to 90° C. During spraying, the coating material and the intake line were heated to 80 to 90° C. in order to obtain a fine spray mist so that an even coating layer formed on the particles and completely enveloped them. During the spray process, the air rate was increased to 60 m³/h, in order to maintain the fluidized-bed height. The spray time was 15 min, the product temperature being 45 to 48° C. and the feed air temperature approximately 45° C. Subsequently, the product was cooled with swirling to 30° C. at 50 m³/h feed air.

A product was obtained having the following characteristic data:

Composition:

| | |
|---|---|
| Corn starch | 68.0% by weight |
| Phytase (dry mass) | 12.0% by weight |
| Polyvinyl alcohol: | 1.1% by weight |
| Zinc sulfate (ZnSO₄): | 0.4% by weight |
| Triglyceride: | 15.0% by weight |
| Residual moisture: | 3.5% by weight |
| Phytase activity: | approximately 11 800 FTU/g |
| Appearance (microscope): | particles having a smooth surface |

### COMPARATIVE EXAMPLE C2

Raw granules were produced analogously to the protocol of comparative example 1, steps a) to c). The resultant raw granules had a phytase activity of approximately 13 000 FTU/g and were subsequently sprayed, in the fluidized-bed apparatus according to comparative example 1, step d), with a commercially available aqueous polyethylene dispersion (solids content 30% by weight, viscosity: 50-300 mPas, pH 9.5-11.5).

For this the raw granules (700 g) were swirled at room temperature with a feed air rate of 35 m³/h. The polyethylene dispersion was sprayed onto the enzyme granules by pumping using a peristaltic pump using a two-fluid nozzle (1.2 mm) at a feed air temperature of 35° C., feed air rate of 45 m³/h, at 1.5 bar. The product temperature during spraying was 30 to 50° C. The dispersion was sprayed onto the enzyme granules in the top-spray method. In this method the water of the dispersion evaporated and the PE particles surrounded the granule particles and stuck to their surface (coating). During spraying, the feed air rate was increased stepwise to 65 m³/h to maintain the swirling. The spraying time was 15 min. Subsequently the product was dried at 30 to 45° C. product temperature for 30 min, the feed air rate being lowered to 55 m³/h in order to keep abrasion of the coating as low as possible.

A product was obtained having the following characteristic data:

Composition:

| | |
|---|---|
| Corn starch | 78.6% by weight |
| Phytase (dry mass) | 12.0% by weight |
| Polyvinyl alcohol: | 1.4% by weight |
| Zinc sulfate (ZnSO₄): | 0.5% by weight |
| Polyethylene: | 4.0% by weight |
| Residual moisture: | 3.5% by weight |
| Phytase activity: | approximately 12 530 FTU/g |
| Appearance (microscope): | particles having a smooth surface |

### EXAMPLE 1

a) In an aqueous phytase concentrate having a dry mass content of about 25-35% by weight, a pH in the range of 3.7-3.9 and an activity of 26 000-36 000 FTU/g, 1% by weight of zinc sulfate hexahydrate, based on the concentrate, was dissolved at 4-10° C. The concentrate was warmed to 30° C. and to it was added 1.1% by weight of methylcellulose (molecular weight of 70 000-120 000 g/mol, viscosity: 4600 cps at 2% by weight in water and 20° C., degree of substitution 1.6-1.9) and stirred until the methylcellulose had completely dissolved. Subsequently, a pH of 5 was set by adding 5% by weight, based on the phytase concentrate, of a 5% strength by weight aqueous ammonia solution.
b) 890 g of corn starch were charged into a mixer having a chopper blade, homogenized and to this were added slowly at temperatures of 10 to 30° C. with homogenization, 433 g of the phytase concentrate from step a). Homogenization was performed with cooling of the mixer for a further 5 min at temperatures in the range from 10 to 50° C., then the resultant dough was transferred to a dome extruder and the dough was extruded at temperatures in the range from 30 to 50° C. through a die having an orifice diameter of 0.7 mm to give 5 cm long strands.
c) The resultant extrudate was rounded in a rounding machine (type P50, from

Glatt) for 5 min. at 350 1/min (rotary speed of the disk) and subsequently dried in a fluidized-bed dryer at a temperature of 40° C. (product temperature) to a residual moisture of about 6% by weight.

The resultant raw granules had an activity of approximately 12 700 FTU/g. The granules had a particle size of a maximum of 1400 μm and a median particle size of 662 μm (sieve analysis).
d) The resultant raw granules were then coated according to the protocol of comparative example C1, step d) in a laboratory fluidized bed Aeromat type MP-1 from Niro-Aeromatic. The coating agent was a commercially available triglyceride based on saturated C₁₆/C₁₈-fatty acids (melting point 57-61° C., iodine value 0.35, saponification value 192).

A product was obtained having the following characteristic data:

Composition:

| | |
|---|---|
| Corn starch | 68.6% by weight |
| Phytase (dry mass) | 12.0% by weight |
| Methylcellulose: | 0.5% by weight |
| Zinc sulfate (ZnSO₄): | 0.4% by weight |
| Triglyceride: | 15.0% by weight |
| Residual moisture: | 3.5% by weight |
| Phytase activity: | approximately 10 450 FTU/g |
| Appearance (microscope): | particles having a smooth surface |

### EXAMPLE 2

Production proceeded analogously to example 1, but in contrast to the protocol stated there, no aqueous ammonia solution was added.

A product was obtained having the following characteristic data:

Composition:

| | |
|---|---|
| Corn starch | 68.6% by weight |
| Phytase (dry mass) | 12% by weight |
| Methylcellulose: | 0.5% by weight |
| Zinc sulfate (ZnSO₄): | 0.4% by weight |
| Triglyceride: | 15.0% by weight |
| Residual moisture: | 3.5% by weight |
| Phytase activity: | approximately 10 760 FTU/g |
| Appearance (microscope): | particles having a smooth surface |

### EXAMPLE 3

The raw granules were produced in an analogous manner to example 1, steps a) to c). The raw granules in this case had a phytase activity of approximately 12 000 FTU/g. The coating was performed analogously to comparative example C2.

A product having the following characteristic data was obtained:

Composition:

| | |
|---|---|
| Corn starch | 79.6% by weight |
| Phytase (dry mass) | 12.0% by weight |
| Methylcellulose: | 0.5% by weight |
| Zinc sulfate (ZnSO₄): | 0.4% by weight |
| Polyethylene: | 4.0% by weight |
| Residual moisture: | 3.5% by weight |
| Phytase activity: | approximately 11 060 FTU/g |
| Appearance (microscope): | particles having a smooth surface |

### Experiment 1 Determination of Pelleting Stability

To assess the pelleting stability of the above-described enzyme granules, a standard pelleting was established. For this, to improve the analytical content determinations, the dosage in the feed was increased. The pelleting was carried out in such a manner that a conditioning temperature of 80 to 85° C. was achieved. Representative samples of the feed before and after pelleting are obtained. The enzyme activity was determined in these samples. If appropriate after correcting for the content of enzyme which is present in the native state, the losses due to pelleting and the relative residual activity (=retention) can be calculated.

The analytical method for phytase is described in various publications: Simple and Rapid Determination of Phytase Activity, Engelen et al., Journal of AOAC International, Vol. 77, No. 3, 1994; Phytase Activity, General Tests and Assays, Food Chemicals Codex (FCC), IV, 1996, p. 808-810; Bestimmung der Phytaseaktivitat in

Enzymstandardmaterialien und Enzympraparaten [Determination of phytase activity in standard enzyme materials and enzyme preparations] VDLUFA-Methodenbuch [Handbook of Methods of the German Association of Agricultural Analytical and Research Institutes], Volume III, 4th supplement 1997; or Bestimmung der Phytaseaktivitat in Futtermitteln und Vormischungen [Determination of phytase activity in feeds and premixes] VDLUFA-Methodenbuch, Volume III, 4th supplement 1997.

As feed, use was made of a conventional broiler feed having the following composition:

| | |
|---|---|
| Corn | 45.5% |
| Soybean extraction meal | 27.0% |
| Full-fat soybeans | 10.0% |
| Peas | 5.0% |
| Tapioca | 4.7% |
| Soybean oil | 3.5% |
| Lime | 1.35% |
| Monocalcium phosphate | 1.30% |
| Cattle salt | 0.35% |
| Vitamin/trace element premix | 1.00% |
| D,L-Methionine | 0.25% |
| L-Lysine HCl | 0.05% |
| | 100% |

The coated granules produced in the above examples were mixed with the above standard feed (content 500 ppm), pelleted and the samples obtained were analyzed. The relative improvement in retention of enzyme activity compared with the granules from comparative example C2 was calculated as follows: Ratio of retention of enzyme activity of the improved granules to retention of enzyme activity of the granules from comparative example C2. The results are summarized in table 1 hereinafter.

TABLE 1
Pelleting stability achieved

| | Description of the granules | Relative pelleting stability |
|---|---|---|
| No. | (coating, binder, pH) | [%] |
| Comparative | 15% fat, 1.1% PVA, pH 3.9 | 118 |
| example C1 | | |
| Comparative | 4% PE, 1.4% PVA, pH 3.9 | 100 |
| example C2 | | |
| Example 1 | 15% fat, 0.5% MC, pH 5 | 147 |
| Example 2 | 15% fat, 0.5% MC, pH 3.9 | 133 |

PE = Polyethylene
PVA = Polyvinyl alcohol
MC = Methylcellulose

1. An enzyme granule for feeds, comprising one or more particles which comprise
A) an enzyme-comprising core having a water content below 15% by weight, based on the weight of the enzyme-comprising core which comprises
i) 50 to 96.9% by weight of at least one solid support material suitable for feeds,
ii) 0.1 to 20% by weight of at least one water-soluble neutral cellulose derivative,
iii) 3 to 49.9% by weight of at least one enzyme,
the percentages by weight of i), ii) and iii) in each case being based on the nonaqueous components of the core; and
B) at least one hydrophobic coating arranged on the surface of the core, which
coating comprises at least one hydrophobic material in an amount of at least 70% by weight, based on the total weight of the coating, selected from waxes, saturated fatty acids, esters of saturated fatty acids, polyolefins or polyamides.

2. The enzyme granule according to claim 1, wherein the weight ratio of core to coating is in the range from 70:30 to 99:1.

3. The enzyme granule according to claim 1, wherein the hydrophobic coating of comprises at least 70% by weight of one or more triglycerides of saturated fatty acids.

4. The enzyme granule according to claim 1, wherein the water-soluble cellulose derivative is methylcellulose.

5. The enzyme granule according to claim 1, wherein the particles have a mean particle size in the range from 100 to 2000 μm.

6. The enzyme granule according to claim 1, wherein the support material comprises at least one water-insoluble polymeric carbohydrate.

7. The enzyme granule according to claim 1, wherein the enzyme is a phosphatase [E.C. 3.1.3].

8. The enzyme granule according to claim 1, wherein the core additionally comprises a salt stabilizing the enzyme in an amount of 0.1 to 10% by weight, based on the total weight of all nonaqueous components of the core.

9. The enzyme granule according to claim 8, wherein the salt is selected from zinc sulfate or magnesium sulfate.

10. A method for producing solid enzyme granules according to claim 1, comprising:
a) providing uncoated, enzyme-comprising raw granules having a water content below 15% by weight, based on the weight of the enzyme-comprising raw granules, and
b) applying the hydrophobic coating to the particles of the raw granules.

11. The method according to claim 10, wherein step a) comprises extrusion of an enzyme-comprising dough which, in addition to water comprises
i) 50 to 96.9% by weight of at least one solid support material suitable for feeds,
ii) 0.1 to 20% by weight of at least one water-soluble neutral cellulose derivative,
iii) 3 to 49.1% by weight of at least one enzyme, the weight fractions of i), ii) and iii) in each case being based on the nonaqueous components of the dough.

12. The method according to claim 10, wherein in step b), the hydrophobic coating is applied in an amount of 1 to 30% by weight, based on the nonaqueous components of the raw granules.

13. The method according to claim 10, wherein in step b), the hydrophobic material is applied in the form of a melt to the particles of the raw granules.

14. The method according to claim 10, wherein in step a), the raw granules, before coating, are subjected to spheronization and subsequently dried to a residual water content of no greater than 15% by weight, based on the total weight of the raw granules.

15. A method of producing feed comprising utilizing the enzyme granules according to claim 1 in feeds.

16. A feed comprising at least one enzyme granule according to claim 1 and customary feed components.

17. The feed according to claim 16 in the form of a pelleted feed.

18. The enzyme granule of claim 7, wherein the phosphatase is a phytase.
